(19) United States
(12) Patent Application Publication
(10) Pub. No.: US 2015/0093760 A1
(43) Pub. Date: Apr. 2, 2015

(54) CARTRIDGE AND SYSTEM FOR DETECTING OF GLYCATED PROTEIN IN SAMPLE AND METHOD OF DETECTING GLYCATED PROTEIN USING THE SAME

(71) Applicant: Samsung Electronics Co., Ltd., Suwon-si (KR)

(72) Inventors: Sang-kyu Kim, Yongin-si (KR); Kyung-mi Song, Suwon-si (KR); Jin-mi Oh, Suwon-si (KR); Youn-suk Choi, Yongin-si (KR)

(21) Appl. No.: 14/503,704

(22) Filed: Oct. 1, 2014

(30) Foreign Application Priority Data
Oct. 1, 2013 (KR) 10-2013-0117590

(51) Int. Cl. G01N 33/72

(52) U.S. Cl. CPC G01N 33/723
USPC 435/7.8; 435/287.1; 436/501; 435/7.1; 435/287.2; 422/69; 436/87; 436/67

(57) ABSTRACT

A cartridge for measuring a concentration of a glycated protein in a wide measurement range, a system for measuring a glycated protein, and a method of measuring a glycated protein using same.

### CROSS-REFERENCE TO RELATED APPLICATIONS

This application claims the benefit of Korean Patent Application No. 10-2013-0117590, filed on Oct. 1, 2013, in the Korean Intellectual Property Office, the disclosure of which is hereby incorporated by reference.

### BACKGROUND

1. Field

The present disclosure relates to a cartridge for efficiently measuring a concentration of a glycated protein in a sample, a system for measuring a concentration of a glycated protein, and a method of measuring a concentration of a glycated protein using the same.

2. Description of the Related Art

A glycated hemoglobin refers to a hemoglobin which is bound to a sugar. An A chain of a hemoglobin may be bound to a sugar. For example, a glycated hemoglobin may include A1a, A1b, A1c, or a combination thereof. Among A1a, A1b, and A1c, hemoglobin A1c (HbA1c), in which a glucose is bound to a valine residue at an N-terminal of a β-chain, has been known to account for about 60% to about 80% of the total glycated hemoglobin.

A glycated hemoglobin may serve as a good indicator of a blood glucose level in a human body because a glycated hemoglobin may show an average blood glucose concentration present in a patient for two to three months prior to measuring the glycated hemoglobin. A conventional method of measuring a glucose level may provide different measurement results depending on whether the measurement has been performed after fasting or after having a meal. However, a method based on a glycated hemoglobin may not be affected by a short-term variation such as food intake.

Accordingly, there has been a need for developing a device for and/or a method of efficiently measuring a concentration of a glycated protein in a sample.

### SUMMARY

An aspect of the present invention provides a cartridge for measuring a concentration of a glycated protein in a blood sample.

Another aspect of the present invention provides a system for measuring a concentration of a glycated protein in a blood sample.

Another aspect of the present invention provides a method of measuring a concentration of a glycated protein in a sample by using the cartridge.

An aspect of the present invention provides a cartridge for measuring a concentration of a glycated protein in a blood sample, wherein the cartridge includes a first chamber and a second chamber, and each chamber is defined by a transparent top plate, a bottom plate, and a spacer between the top plate and the bottom plate, and wherein a surface of the top plate or the bottom plate includes a glycated protein-binding substance and a buffer.

The term “glycated protein” may include a glycated polypeptide or a glycated amino acid. A “glycated protein” may be, for example, a glycated hemoglobin, a fragment of a glycated hemoglobin, a glycated amino acid, or a combination thereof. The glycated hemoglobin may include A1a, A1b, A1c, or a combination thereof.

In the cartridge, the glycated protein-binding substance may be arranged on a surface of the top plate and/or the bottom plate. The glycated protein-binding substance may be an antibody, boronic acid, concanavalin, or a combination thereof. The antibody may be a whole antibody, an antibody fragment, polyfunctional antibody aggregate, or a combination thereof. The antibody may be an anti-hemoglobin antibody or an anti-glycated hemoglobin antibody.

The glycated protein-binding substance (i.e., a substance capable of binding a glycated protein) may further include 3-[(3-cholamidopropyl)-dimethylammonio]-1-propane sulfonate (CHAPS) and/or sorbitol. The content of the CHAPS may be in a range from about 0.1% to about 0.5%, from about 0.1% to about 0.4%, from about 0.1% to about 0.3%, from about 0.1% to about 0.2%, or from about 0.15% to about 0.2%. The content of the sorbitol may be in a range from about 1% to about 15%, from about 2% to about 14%, from about 3% to about 13%, from about 4% to about 12%, from about 5% to about 11%, from about 6% to about 10%, or from about 7% to about 9%.

In addition, the glycated protein-binding substance may include a detectable marker. The marker may be, for example, a marker generating an optical signal, a radioactive marker, or a marker generating an electric signal. The marker may be, for example, a fluorescent substance generating a fluorescent signal. The fluorescent substance may be Cal610, fluorescein, rhodamine, a cyanine such as Cy3 and Cy5, or a metal porphyrin complex. The glycated protein-binding substance may be a dried substance.

Each of the first and second chambers (or additional chambers, if present) may be designed to detect or measure different concentrations of a glycated protein. For instance, the first chamber may be a chamber to measure a glycated protein concentration lower than that of the second chamber. A pre-determined concentration difference between the glycated protein-binding substance and the buffer may optimize each chamber to be a chamber for measuring a different glycated protein concentration. According to an embodiment of the present invention, the first chamber and the second chamber may include the same glycated protein-binding substance (e.g., same glycated protein-binding antibody) and the concentration of the glycated protein-binding substance in the first chamber may be lower than that of the second chamber. Or, the first chamber and the second chamber may include the same buffer, and the concentration of the buffer in the first chamber may be higher than that of the second chamber. Also, the first chamber and the second chamber may include the same glycated protein-binding substance and the same buffer, wherein the concentration of the glycated protein-binding substance in the first chamber may be lower than that of the second chamber, and the concentration of the buffer in the first chamber may be higher than that of the second chamber.

The buffer may be, for example, phosphoric acid, carbonic acid, an organic acid buffer, or Good's buffer. An acid may present to control pH of a solution including a buffer and the acid may be, for example, an inorganic acid such as hydrochloric acid, or an organic acid such as acetic acid. In addition, a base may be present to control the pH of the solution including a buffer, and the base may be sodium hydroxide, potassium hydroxide, lithium hydroxide, or ammonium hydroxide. In addition, the buffer may be a non-ionic surfactant having a polyoxyethylene glycol group, a cationic surfactant, or an anionic surfactant.

The buffer may further include other additives, such as bovine serum albumin (BSA), polyethylene glycol (PEG), casein, or a combination thereof.

In the cartridge, the concentration of the antibody such as anti-hemoglobin antibody or an anti-glycated hemoglobin antibody may be present in a range from about 1 μg/ml to about 1000 μg/ml. The concentration of the additive may be in a range from about 0.01 mg/ml to about 1000 mg/ml.

In the cartridge, the height of the spacer (dimension separating the top plate from the bottom plate) may be in a range from about 1 μm to about 1000 μm, for example, from about 1 μm to about 500 μm, from about 1 μm to about 300 μm, from about 1 μm to about 200 μm, from about 1 μm to about 100 μm, or from about 1 μm to about 10 μm. The cartridge may be prepared in a micrometer dimension (e.g., less than 1000 microns in thickness including the top plate and the bottom plate) to be used in a miniaturized light absorption apparatus having a short optical path.

In the cartridge, the top plate and the bottom plate may be transparent. In addition, the top plate and the bottom plate may be a film. The top plate and the bottom plate may be a polyethylene terephtalate (PET) film, a polyethylene (PE) film, a polypropylene film (PP), a polyvinylchloride (PVC) film, a polyvinyl alcohol (PVA) film, or a polystyrene (PS) film.

Another aspect of the present invention provides a system for measuring a concentration of a glycated protein, including a cartridge for measuring a concentration of a glycated protein, wherein the cartridge includes a first chamber and a second chamber, wherein the cartridge includes a transparent top plate, a bottom plate, and a spacer between the top plate and the bottom plate, and the chamber is surrounded by the top plate, the bottom plate, and the spacer, and wherein a surface of the top plate or the bottom plate includes a glycated protein-binding substance, a buffer, and an additive; a storage part including (storing) a mathematical formula representing a predetermined calibration curve included in the first chamber and the second chamber (and other chambers if present); a measurement part that measures a signal produced by the binding of a glycated protein to the substance to glycated protein in a sample introduced to the first and/or second chambers; and a determination part that determines or measures the concentration of a glycated protein by using the mathematical formula of the calibration curve. The predetermined calibration curve may be obtained by measuring known concentration of glycated proteins (e.g., HbA1c concentration of range between about 2% and about 20%) by the cartridge. When a signal measured in the first chamber is equal to or less than a predetermined threshold, the determination part measures the concentration of glycated protein in the first chamber using the mathematical formula representing the calibration curve for the first chamber. When a signal measured in the first chamber exceeds a predetermined threshold, the determination part measures the concentration of glycated protein in the second chamber using the mathematical formula of the calibration curve for the second chamber.

The measurement part measures a signal of glycated protein bound to the glycated protein binding substance in the first chamber or the second chamber. Signal measurement may be measurement of an optical signal, an electric signal, a mechanical signal, or a combination thereof. The signal measurement may be a measurement of, for example, an optical signal (e.g., absorbance) specific to a glycated protein or fluorescence. The signal measurement may be a measurement of an absorbance specific to a glycated protein, for example, a measurement of an absorbance in a wavelength range from about 400 nm to about 700 nm.

The storage, measurement, and determination parts each, respectively, stores a calibration curve, measures a signal such as an absorbance, and measures a concentration of glycated protein through a predetermined scenario by means of a software program.

The mathematical formula of the calibration curve may be obtained by using a calibrator with various known concentrations of glycated proteins. A calibrator including glycated proteins having different known concentrations is used to measure a signal, calculate a linear correlation coefficient (R) between the concentration of the glycated protein and the signal, and, as a result, obtain the mathematical formula of the calibration curve. The mathematical formula of the calibration curve may have a linear correlation coefficient (R) in a range of, for example, 0.97 or higher, 0.98 or higher, 0.99 or higher, or from about 0.97 to about 1.0000, from about 0.98 to about 0.9990, from about 0.99 to about 0.9980, from about 0.99 to about 0.9970, from about 0.99 to about 0.9965, from about 0.99 to about 0.9960, from about 0.99 to about 0.9955, from about 0.99 to about 0.9950, from about 0.99 to about 0.9945, from about 0.99 to about 0.9940, or from about 0.99 to about 0.9930.

A predetermined threshold may be, in a concentration range of a glycated protein used to obtain a calibration curve obtained in the first chamber, a signal corresponding to a highest concentration of the glycated protein on the calibration curve, for example, the highest concentration of the glycated protein of the calibrator, or some other selected concentration value. The signal corresponding to the concentration of the glycated protein may include a signal having a signal error range from about ±0.001 to about ±0.010, from about ±0.001 to about ±0.009, from about ±0.001 to about ±0.008, from about ±0.001 to about ±0.007, from about ±0.001 to about ±0.006, from about ±0.001 to about ±0.005, from about ±0.001 to about ±0.004, from about ±0.001 to about ±0.003, or from about ±0.001 to about ±0.002.

The calibration curve may be used to perform a conversion of the signal of the glycated protein measured in a blood sample to the concentration of the glycated protein. The calibration curve may represent, for example, a logarithmic function, an exponential function, a sigmoid function, or a linear function. During the conversion, as the logarithmic function has a high discrimination power with respect to the low range of the concentration of the glycated protein, the logarithmic function may convert the signal to the concentration at a high accuracy when low concentrations are present. However, as the logarithmic function has a low discrimination power with respect to the high range of the concentration of the glycated protein, the logarithmic function may convert the signal to the concentration at a low accuracy when high concentrations are present. The exponential function has a high discrimination power with respect to the high range of the concentration of the glycated protein, and the exponential function may convert the signal to the concentration at a high accuracy when high concentrations are present. However, as the exponential function has a low discrimination power with respect to the low range of the concentration of the glycated protein, the exponential function may convert the signal to the concentration at a low accuracy when low concentrations are present. In addition, the during the conversion, as the sigmoid function has a high discrimination power with respect to the medium range of the concentration of the glycated protein, the sigmoid function may convert the signal to the concentration at a high accuracy when medium concentrations are present. However, as the sigmoid function has a low discrimination power with respect to the low range and the high range of the concentration of the glycated protein, the sigmoid function may convert the signal to the concentration at a low accuracy when low or high concentrations are present. The linear function has a high discrimination power with respect to all ranges of the concentration of the glycated protein; the linear function may convert the signal to the concentration at a high accuracy.

Another aspect of the present invention provides a method of measuring a concentration of a glycated protein, the method including storing multiple glycated protein-binding substances and buffers respectively in a first chamber and a second chamber of a cartridge; introducing blood samples into the chambers; and measuring signals from the individual chambers; and determining the concentration of a glycated protein by using the mathematical formula of a calibration curve for the first chamber if a signal measured in the first chamber is equal to or less than a predetermined threshold, or determining the concentration of a glycated protein by using the mathematical formula of a calibration curve for the second chamber in a case where a signal measured in the first chamber exceeds a predetermined threshold.

The blood sample may be whole blood cells, collected blood cells, or hemolyzed blood. In addition, the sample may be a sample including cells, a sample including tissues, or a combination thereof.

The method may further include dissolving of a blood sample of which a concentration of a glycated protein is to be measured before a reaction. A lysing buffer may be used to separate a glycated protein in a red blood cell from a blood sample to be measured. The lysing buffer may be deionized water (DW), a zwitterionic surfactant, an anionic surfactant, a cationic surfactant, a neutral surfactant, or a combination thereof. The lysing buffer may be a Triton such as Triton X-100, a Tween such as a Tween 20, or a detergent such as sodium dodecyl sulfate (SDS), cetyltrimethylammonium bromide (CTAB), tetradecyltrimethylammonium bromide (TTAB), polyoxyethylene lauryl ether (POE), and Nonidet P-40.

The method may provide adding an insoluble carrier particle to a lysed blood sample. The insoluble carrier particle may be selected from the group consisting of a latex particle, a gold nanoparticle, an agarose particle, a sepharose, a glass particle, and a combination thereof. The particle may be a bead. The particle may be nonspecifically adsorbed to a glycated protein through physical and/or chemical adsorption.

The insoluble carrier particle may be also, for example, a synthetic resin (latex) such as polystyrene, polyvinylchloride, polypropylene, (meta) acrylic resin, polymethylmethacrylate; a cellulose derivative such as nitrocellulose, cellulose, and methylcellulose; or an inorganic material such as a metal, ceramic, glass, and silicon rubber. When a latex with a hydrophobic surface is used, a protein or a peptide may be adsorbed to the surface. In addition, the latex may include a denatured latex such as carboxyl-denatured latex or a latex in which a magnetic particle is impregnated. The shaped of the insoluble carrier particle may be, for example, spherical. The mean diameter of the spherical particle may be in a range, for example, from about 0.03 μm to about 0.8 μm, for example, from about 0.06 μm to about 0.2 μm.

The method includes measuring a signal of the reaction product. As a glycated protein is bound to a glycated protein-binding substance and thus the size of aggregate of the glycated protein and the glycated protein-binding substance is increased, measurement of the reaction product shows a change in absorption wavelength or an increase or decrease of absorbance. As the size of the aggregate is increased in proportion to the glycated protein concentration in blood, an extent of the increase of the aggregate formation is measured and quantified to measure the concentration of the glycated protein in a blood sample. Measuring of absorbance may be measuring of light absorbance in a wavelength range from about 400 nm to about 700 nm.

The method may further include storing a predetermined calibration curve with respect to each reaction reagent included in the first chamber and the second chamber.

The method may further include measuring a concentration of a glycated protein by using the calibration curve obtained in the first chamber in a case where a signal measured in the first chamber is equal to or less than a predetermined threshold, or measuring a concentration of a glycated protein by using the calibration curve obtained in the second chamber in a case where a signal measured in the first chamber exceeds a predetermined threshold. The predetermined threshold is described above.

### BRIEF DESCRIPTION OF THE DRAWINGS

These and/or other aspects will become apparent and more readily appreciated from the following description of the embodiments, taken in conjunction with the accompanying drawings in which:

FIG. 1 is a diagram depicting a cartridge for measuring a glycated protein;

FIG. 2 is a diagram depicting a cartridge for measuring a glycated protein including multiple chambers;

FIG. 3 is a sectional view of a cartridge for measuring a glycated protein;

FIG. 4 is a graph depicting the effect of the concentration of an antibody;

FIG. 5 is a graph depicting the effect of the concentration of a buffer on the measurement of glycated protein concentration;

FIG. 6 is a diagram depicting a scenario for measuring a concentration of a glycated protein from two calibration curves;

FIG. 7 is a diagram depicting a calibration curve in a case where a cartridge including a single chamber was used to measure the concentration of the glycated protein and the accuracy of the concentration of the glycated protein calculated by using the calibration curve; and

FIG. 8 depicts two calibration curves obtained in a case when a cartridge including multiple chambers coated with reagents under different conditions was used to measure the glycated protein concentration.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items.

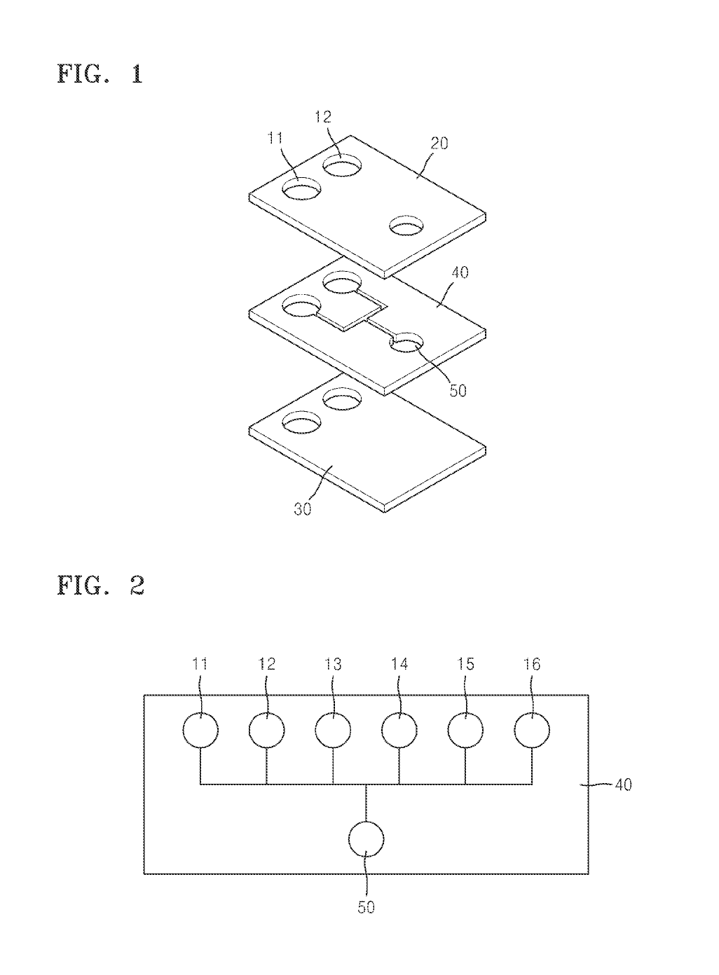

FIGS. 1 through 3 are diagrams depicting a cartridge according an embodiment of the present invention.

As shown in FIG. 1, a cartridge for measuring a glycated protein may include a first chamber (11) and a second chamber (12). The chamber may be an elliptical chamber having a pattern shown in FIG. 1. The cartridge may include a top plate (20), a bottom plate (30), and a spacer (40) between the top plate (20) and the bottom plate (30), which define the chambers (11) and (12). The top plate (20) and the bottom plate (30) may be made of a transparent material. The top plate (20) and the bottom plate (30) may be made of a polyethylene terephthalate film. The spacer (40) may be made of a cellulose acetate membrane. An inlet (50) may be located on the top plate and the spacer. After the top plate (20), the bottom plate (30), and the spacer (40) are assembled, a sample may be introduced through the inlet (50) to the first chamber (11) and the second chamber (12) of the cartridge. The inlet (50) may be an inlet without a filter. A blood sample introduced through the inlet may dissolve a dried reagent located on the top plate or the bottom plate of the cartridge to induce a reaction by diffusion. Thus, the cartridge does not need a separate mixing apparatus. The thickness of the spacer may be from about 1 um to about 1000 um.

FIG. 2 is a diagram depicting a cartridge for measuring a glycated protein, including multiple chambers according to an embodiment of the present invention. As shown in FIG. 2, the cartridge may be a cartridge including multiple chambers, for example, a first chamber (11), a second chamber (12), a third chamber (13), a fourth chamber (14), a fifth chamber (15), and a sixth chamber (16).

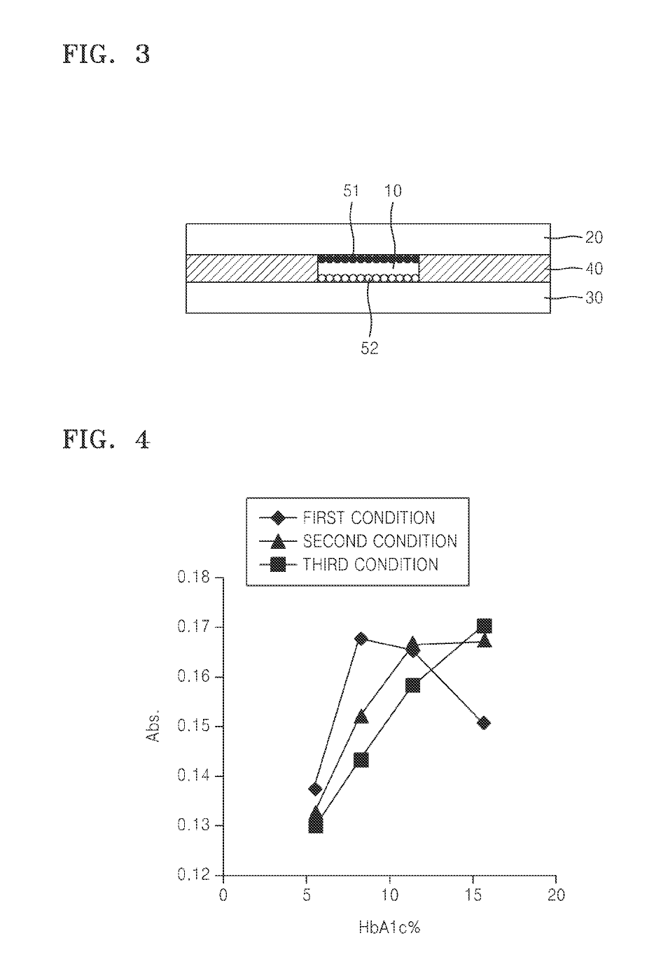

FIG. 3 shows a sectional view of a cartridge for measuring a glycated protein according to an embodiment of the present invention. As shown in FIG. 3, a glycated protein-binding substance (51) such as an antibody, and a buffer (52) may be included in a chamber (10) formed by assembling a top plate (20), a bottom plate (30), and a spacer (40). The glycated protein-binding substance (51), and the buffer (52) may be coated on a surface of the top plate (20) and/or the bottom plate (30) toward the chamber (10) and stored in a dried state. The buffer may include an additive, such as bovine serum albumin (BSA), polyethylene glycol (PEG), casein, or a combination thereof. A region of the top plate (20) and of the bottom plate (30) which is not corresponding to the chamber (10) may be coated as opaque. The region may be coated by a screen printing method. The region may be coated with a shading ink. The coating with a shading ink my protect substances inside the chamber from external light or prevent an error in glycated protein signal measurement, for example, an error in optical signal measurement.

FIG. 6 is a diagram showing a scenario for measuring a concentration of a glycated protein from two calibration curves according to an embodiment of the present invention. A reaction reagent with a low antibody concentration and a high buffer concentration is stored in the first chamber to accurately measure a glycated protein of a low concentration. In addition, a reaction reagent with a high antibody concentration and a low buffer concentration is stored in the second chamber to accurately measure a glycated protein of a high concentration. A calibration curve may be obtained for each reagent/chamber.

As shown in FIG. 6, after introducing a blood sample to the cartridge, absorbance in the first chamber (OD1) and absorbance in the second chamber (OD2) may be measured. OD1 and a predetermined threshold are compared, when OD1 is equal to or less than the threshold, the calibration curve obtained in the first chamber (Cal. 1) is used to measure a concentration of a glycated protein.

The predetermined threshold may be, for instance, in a concentration range in which a correlation coefficient of a calibration curve obtained in the first chamber, R, is 0.99 or higher. As a further example, the predetermined threshold may be an absorbance corresponding to a highest concentration of the glycated protein, for example, an absorbance corresponding to a concentration of a glycated protein in a calibrator. The absorbance corresponding to the concentration of the glycated protein may include a signal having a signal error range from about ±0.001 to about ±0.010, from about ±0.001 to about ±0.009, from about ±0.001 to about ±0.008, from about ±0.001 to about ±0.007, from about ±0.001 to about ±0.006, from about ±0.001 to about ±0.005, from about ±0.001 to about ±0.004, from about ±0.001 to about ±0.003, or from about ±0.001 to about ±0.002. When the OD1 exceeds the predetermined threshold, the measurement signal and calibration curve corresponding to the second chamber (Cal. 2) is used to measure a concentration of a glycated protein. As described above, through a scenario determined in advance, an optimal calibration curve may be selected to measure an accurate concentration of a glycated protein. In addition, the concentration of an antibody, a buffer, and an additive may be controlled so that the measurement condition may have a good discrimination power with respect to the low range of the concentration, a good discrimination power with respect to the high range of the concentration or a combination thereof. As a result, the cartridge may be prepared to have a wide measurement range. Reagents of multiple conditions may be respectively stored in a cartridge including multiple chambers to prepare a glycated protein having a wide measurement range.

### Example 1

### Preparation of Cartridge for Measuring Glycated Hemoglobin

A top plate and a bottom plate of a cartridge were made of a patterned polyethylene terephthalate film. A spacer arranged between the top plate and the bottom plate was made of a patterned cellulose acetate membrane. The membrane was water-repellent treated so that a fluid might flow through a flow path and the air is discharged through the membrane. On the top plate film, a solution including a glycated hemoglobin-binding substance including an antibody (hereinafter referred to as “R2 solution”) was coated. The R2 solution was concentrated under two conditions and 0.25 μl of the R2 solution including a first condition antibody was coated on a first chamber and 0.25 μl of the R2 solution including a second condition antibody was coated on a second chamber. The first condition was to prepare the R2 solution including 120 ng/ml of anti-HbA1c, 40 ng/ml anti-IgG, 0.2% of CHAPS, and 8% of sorbitol in 0.3×PBS buffer. The second condition was to prepare the R2 solution including 160 ng/ml of anti-HbA1c, 50 ng/ml anti-IgG, 0.2% of CHAPS, and 8% of sorbitol in 0.1×PBS buffer. A chip on which the reagents were coated were dried under 1% humidity condition for one night and the top plate, the bottom plate, and a space were assembled to be used.

### Example 2

### Obtaining Glycated Hemoglobin Calibration Curve when Antibody or Buffer Concentrations are Different

(2.1) Glycated Hemoglobin Calibration Curve when Antibody Concentrations are Different

In the multiple chambers of the cartridge prepared in Example 1, antibodies of a concentration under different conditions including a first condition, a second condition, and a third condition were coated and stored in a dried state. A monoclonal antibody, anti-HbA1c, was used. Calibrators (Cliniqa, US) having four different concentrations were mixed with a solution including both a lysed blood and a bead (hereinafter referred to as “R1 solution”) and the resulting solution was introduced through an inlet of the cartridge to obtain three glycated hemoglobin calibration curves. The bead was a latex bead. The latex bead was prepared by concentrating an HbA1c kit R1 solution (Fujirebio) by about four times.

FIG. 4 is a diagram depicting the effect of the concentration of an antibody according to an embodiment of the present invention on the measurement of glycated protein concentration. As antibody concentration was 80 μg/ml (first condition), 120 μg/ml (second condition), and 160 μg/ml (third condition), the concentration was the lowest under the first condition and the highest under the third condition. As shown in FIG. 4, as the antibody concentration is higher with respect to the blood sample including an adsorbed bead, the measurement range is wider, while the signal discrimination power is lower depending on the HbA1c concentration. Additionally, as the antibody concentration is lower, the measurement range is narrower, while the signal discrimination power is higher in a low concentration interval.

(2.1) Glycated Hemoglobin Calibration Curve when Antibody Concentrations are Different

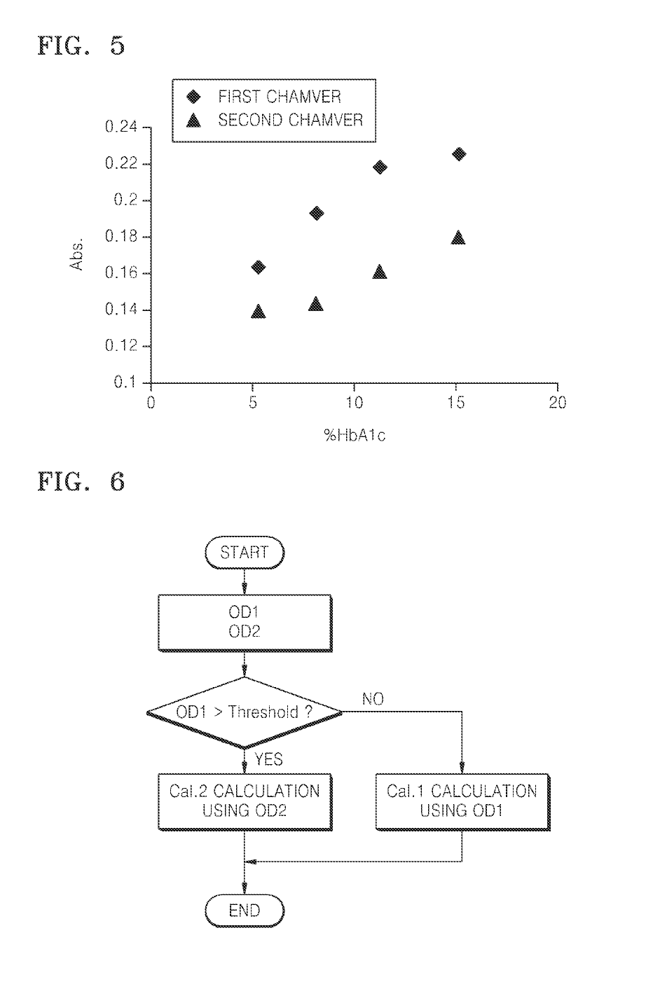

Among the multiple chambers of the cartridge prepared in Example 1, buffers of a concentration under different conditions were coated in a first chamber and a second chamber and stored in a dried state. The antibody concentration was the same in this case. As in the case of Example 2.1, Calibrators (Cliniqa, US) having four different concentrations were mixed with both a lysed blood and a bead and the resulting solution was introduced through an inlet of the cartridge to obtain two glycated hemoglobin calibration curves. FIG. 5 is a diagram depicting the effect of the concentration of a buffer according to an embodiment of the present invention on the measurement of glycated protein concentration. The concentration of the buffer in the first chamber was higher than that of the buffer in the second chamber. As shown in FIG. 5, as the buffer concentration is higher, the discrimination power becomes better in a low glycated hemoglobin concentration and, as the buffer concentration is higher, the discrimination power becomes better in a high glycated hemoglobin concentration.

### Example 3

### HbA1c Measurement Using Latex Coagulation Reaction

A calibrator (Cliniqa, US) having four different concentrations was mixed with a hemolytic solution (prepared by performing hemolysis by mixing 1 μl of a calibrator and 200 μl deionized water) and the R1 solution described in Example 1 and the resulting solution was introduced to the cartridge prepared in Example 1 to mix the resulting solution with the R2 reagent coated on the cartridge and observe by using LABGEO PT10 (Samsung Electronics) the variation of absorbance according to a coagulation reaction.

With respect to the four calibrators introduced to the cartridge, the absorbance was measured by using Tosoh G8. The concentration of HbA1c calculated from the measured absorbance was 5.3%, 8.1%, 11.2%, and 15.1%, respectively.

(3.1) Measurement of Glycated Hemoglobin Concentration Using Cartridge Including Single Chamber

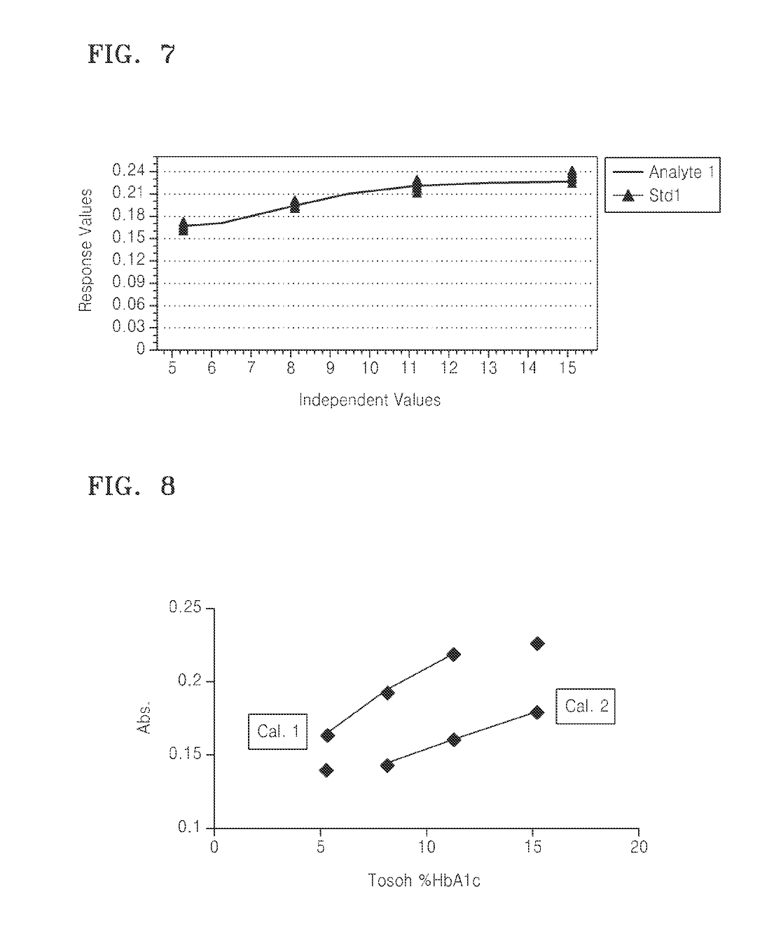

Four calibrators were introduced to a cartridge including a single chamber to measure a concentration of a glycated hemoglobin. FIG. 7 is a diagram depicting a calibration curve in a case when a cartridge including a single chamber and a total 0.25 μl of the R2 solution (120 ng/ml of anti-HbA1c, 40 ng/ml anti-IgG, 0.2% of CHAPS, and 8% of sorbitol in 0.3×PBS buffer) including the reagent under the first condition were used to measure the concentration of the glycated hemoglobin.

A signal processing software program MasterPlex (Hitachi Solutions) was used to process the signal measured in the single chamber and to obtain a single calibration curve in a best fit mode. FIG. 7 shows the calibration curve represented by the MasterPlex (Hitachi Solutions) by setting the calibrator concentration to be an independent value and the absorbance to be a response value. The obtained calibration curve is a four-parameter logistic curve having an R value of 0.988666, an RMSE value of 0.00380, an a value of 0.16028, a b value of 6.29760, a c value of 8.11355, and a d value of 0.22767. As shown in FIG. 7, the signal measured in the single chamber coated with the reagent under the first condition showed a sigmoid calibration curve

Table 1 shows the accuracy of the measurements of the glycated hemoglobin concentration obtained by using the cartridge including a single chamber. As shown in Table 1, the CV of Lv1, which was a sample of a low glycated hemoglobin concentration, was as high as 9.2%, while the CV of Lv4, which was a sample of a high glycated hemoglobin concentration, was as high as 4.9%.

In addition, although not shown in FIG. 7, signals of a number of samples for the Lv1 and Lv4 calibrators were not able to be converted. In addition, in the Lv3 and Lv4 calibrators, the signals for measuring the concentration of the glycated hemoglobin were overlapped with each other so that the concentration obtained through the calibration curve shown in FIG. 7 might provide twisted information.

TABLE 1

| | Lv1 | Lv 2 | Lv 3 | Lv 4 |
|---|---|---|---|---|
| CV | 9.2% | 2.4% | 6.4% | 4.9% |

(3.2) Measurement of Glycated Hemoglobin Concentration Using Cartridge Including Multiple Chambers Coated with Reagents Under Different Conditions According to an Embodiment of the Present Invention

Two chambers coated with different reagents under a first condition and under a second condition were used. The four calibrators the same as those used in Example 3 were introduced and the glycated hemoglobin concentration was measured to obtain two calibration curves. The reagent under the first condition was 0.25 μl of the R2 solution including 120 ng/ml of anti-HbA1c, 40 ng/ml anti-IgG, 0.2% of CHAPS, and 8% of sorbitol in 0.3×PBS buffer and the reagent under the second condition was 0.25 μl of the R2 solution including 160 ng/ml of anti-HbA1c, 50 ng/ml anti-IgG, 0.2% of CHAPS, and 8% of sorbitol in 0.1×PBS buffer.

FIG. 8 shows the two calibration curves obtained from the measured glycated hemoglobin concentration in the case when a cartridge including multiple chambers coated with reagents under different conditions according to an embodiment of the present invention was used to measure the glycated hemoglobin concentration. FIG. 8 shows the glycated hemoglobin concentration in each chamber obtained through measurement of the absorbance. The linear correlation coefficient between the signal measured in the first chamber and the glycated hemoglobin concentration was calculated to obtain the mathematical formula of the calibration curve. In the obtained mathematical formula of the calibration curve, the concentration interval in which the correlation coefficient was about 0.99 or higher was searched. The calibration curve obtained from the first chamber was obtained in the glycated hemoglobin concentration interval of the Lv1 through Lv3 calibrators and the obtained calibration curve was named as Cal.1. Among the calibrators used for Cal.1, Lv3 showed the highest glycated hemoglobin concentration and the absorbance of Lv3 was about 0.22.

In the same manner, the linear correlation coefficient between the signal measured in the second chamber and the glycated hemoglobin concentration was calculated to obtain the mathematical formula of the calibration curve. In the obtained mathematical formula of the calibration curve, the concentration interval in which the correlation coefficient was about 0.99 or higher was searched. The calibration curve obtained from the second chamber was obtained in the glycated hemoglobin concentration interval of the Lv2 through Lv4 calibrators and the obtained calibration curve was named as Cal.2.

The correlation between the absorbance and the HbA1c concentration (%) in the obtained Cal.1 was y=0.0734x+0.0416 (y denotes the absorbance and x denotes the HbA1c concentration (%).) and the correlation coefficient R² was 0.998 (R was 0.9989.). The correlation between the absorbance and the HbA1c concentration (%) in Cal.2 was y=0.0051x+0.1038 (y denotes the absorbance and x denotes the HbA1c concentration (%).) and the correlation coefficient R² was 0.9975 (R was 0.9987.). Cal.1, which was a logarithmic curve, showed a high discrimination power at a low concentration, while Cal.2, which was a linear curve, showed a good discrimination power in Lv2 through Lv4 interval. Therefore, the two calibration curves were combined to calculate the glycated hemoglobin concentration in the calibrators.

Among the calibrators used in Example 4, the glycated hemoglobin concentration of Lv1 through Lv3 calibrators was calculated by using Cal.1, while the glycated hemoglobin concentration of Lv4 calibrator was calculated by using Cal.2. Among the used calibrators, the glycated hemoglobin concentration was already known and thus the glycated hemoglobin concentration obtained by the glycated hemoglobin concentration calculation method using the two calibration curves, Cal.1 and Cal.2, was compared with the actual glycated hemoglobin concentration.

Table 2 shows the accuracy of the glycated hemoglobin concentration measurements obtained by combining the two calibration curves. As shown in Table 2, the accuracy of the glycated hemoglobin concentration measurements obtained by combining the two calibration curves was higher than that obtained the glycated hemoglobin concentration measurements obtained by using a single calibration curve.

TABLE 2

| | Cal1 | | | Cal2 |
|---|---|---|---|---|
| | Lv 1 | Lv 2 | Lv 3 | Lv 4 |
| CV | 3.4% | 3.3% | 4.4% | 2.9% |

As described above, according to a cartridge according to an aspect of the present invention, a concentration of a glycated protein may be accurately measured and a glycated protein may be measured in a wide measurement range.

According to a system for measuring a glycated protein according to an aspect of the present invention, a glycated protein may be measured in a wide measurement range.

According to a method of measuring a glycated protein according to an aspect of the present invention, a concentration of a glycated protein may be accurately measured and a glycated protein may be measured in a wide measurement range.

It should be understood that the exemplary embodiments described therein should be considered in a descriptive sense only and not for purposes of limitation. Descriptions of features or aspects within each embodiment should typically be considered as available for other similar features or aspects in other embodiments.

While one or more embodiments of the present invention have been described with reference to the figures, it will be understood by those of ordinary skill in the art that various changes in form and details may be made therein without departing from the spirit and scope of the present invention as defined by the following claims.

What is claimed is:

1. A cartridge for measuring the concentration of a glycated protein in a sample, wherein the cartridge comprises a first chamber and a second chamber and wherein the first chamber and the second chamber comprise a glycated protein-binding substance and a buffer.

2. The cartridge of claim 1, wherein the first chamber and the second chamber comprise a same glycated protein-binding substance, and wherein the glycated protein-binding substance has a concentration in the first chamber that is lower than the concentration of the glycated protein-binding substance in the second chamber.

3. The cartridge of claim 1, wherein the first chamber and the second chamber comprise a buffer, and wherein the concentration of the buffer in the first chamber is higher than that in the second chamber.

4. The cartridge of claim 1, wherein the cartridge comprises a top plate, a bottom plate, and a spacer between the top plate and the bottom plate, and the chamber is surrounded by the top plate, the bottom plate, and the spacer.

5. The cartridge of claim 4, wherein a surface of the top plate or bottom plate in the first chamber and the second chamber comprise the same glycated protein-binding substance, and the surface of the top plate or bottom plate in the first chamber and the second chamber comprise a buffer, wherein the concentration of the glycated protein-binding substance on the surface of the top or bottom plate in the first chamber is lower than the concentration of the glycated protein-binding substance on the surface of the top or bottom plate in the second chamber, and wherein the concentration of the buffer on the top or bottom plate in the first chamber is higher than the concentration of the buffer on the top or bottom plate in the second chamber.

6. The cartridge of claim 2, wherein the glycated protein-binding substance is an antibody, boronic acid, concanavalin, or a combination thereof.

7. The cartridge of claim 2, wherein the glycated protein-binding substance comprises 3-[(3-cholamidopropyl)-dimethylammonio]-1-propane sulfonate (CHAPS) or sorbitol.

8. The cartridge of claim 3, wherein the buffer comprises an additive, and the additive is bovine serum albumin (BSA), polyethylene glycol (PEG), casein, or a combination thereof.

9. The cartridge of claim 6, wherein the glycated protein-binding substance is an antibody, and the concentration of the antibody is about 1 μg/ml to about 1000 μg/ml.

10. The cartridge of claim 8, wherein the concentration of the additive is about 0.01 mg/ml to about 1000 mg/ml.

11. The cartridge of claim 1, wherein the glycated protein-binding substance and the buffer are dried substances.

12. The cartridge of claim 4, wherein the height of the spacer is about 1 μm to about 1000 μm.

13. A system for measuring a concentration of a glycated protein in a blood sample, comprising a cartridge of claim 1 for measuring the concentration of a glycated protein in a sample;
a storage part storing a predetermined calibration curve with respect to each reaction reagent included in the first chamber and the second chamber;
a measurement part that measures a signal of a glycated protein bound to the glycated protein-binding substance in the first chamber and the second chamber; and
a determination part that measures the concentration of a glycated protein using the calibration curve for the reagents of the first chamber if the signal measured in the first chamber is equal to or less than a predetermined threshold, or using the calibration curve for the reagents in the second chamber in a case if the signal measured in the first chamber exceeds a predetermined threshold concentration.

14. A method of measuring a concentration of a glycated protein comprising storing a glycated protein-binding substance and buffer in a first chamber and a second chamber of a cartridge;
injecting a blood sample into the chambers;
measuring a signal from the first chamber and second chamber; and
determining the concentration of glycated protein by comparing the signal from the first or second chamber to using a pre-determined calibration curve, wherein, if the signal measured in the first chamber is equal to or less than a predetermined threshold, the concentration of glycated protein is determined using the signal from the first chamber and pre-determined calibration curve from the first chamber, and if the signal measured in the first chamber exceeds a predetermined threshold, the concentration of glycated protein is determined using the signal from the second chamber and pre-determined calibration curve from the second chamber.

15. The method of 14, wherein the predetermined threshold is, in a concentration range of a glycated protein used to obtain a calibration curve obtained in the first chamber, a signal corresponding to a highest concentration of the glycated protein.

16. The method of claim 14, wherein the first chamber and the second chamber comprise the same glycated protein-binding substance, and wherein the glycated protein-binding substance has a concentration in the first chamber that is lower than the concentration of the glycated protein-binding substance in the second chamber.

17. The method of claim 14, wherein the first chamber and the second chamber comprise a buffer, and wherein the concentration of the buffer in the first chamber is higher than that in the second chamber.

18. The method of claim 14, wherein the cartridge comprises a top plate, a bottom plate, and a spacer between the top plate and the bottom plate, and the chamber is surrounded by the top plate, the bottom plate, and the spacer.

19. The method of claim 14, wherein the method further comprises adding a lysing buffer and an insoluble carrier particle to the blood sample.

20. The method of claim 19, wherein the insoluble carrier particle is a latex particle, a gold nanoparticle, an agarose particle, a sepharose, a glass particle, or a combination thereof.
